(19) United States
(12) Patent Application Publication
(10) Pub. No.: US 2026/0305636 A1
(43) Pub. Date: Oct. 8, 2026

(54) METHODS FOR PRODUCING CONTINENTAL TYPE CHEESES

(71) Applicant: CHR. HANSEN A/S, Hoersholm (DK)

(72) Inventors: PETER BEIERHOLM POULSEN, Hvidovre (DK); Morten CARLSON, Ramloese (DK); Sebastien ROUSTEL, Chalon (FR)

(73) Assignee: CHR. HANSEN A/S, Hoersholm (DK)

(21) Appl. No.: 19/484,979

(22) PCT Filed: May 29, 2024
(86) PCT No.: PCT/EP2024/064722

§ 371 Date: Nov. 17, 2025

(30) Foreign Application Priority Data
May 30, 2023 (EP) 23176053.9

(51) Int. Cl. A23C 19/032; A23C 19/068; C12N 1/20

(52) U.S. Cl. CPC A23C 19/0323; A23C 19/0688; C12N 1/20; A23V 2400/215; A23V 2400/231; A23V 2400/249

(57) ABSTRACT

The present disclosure relates to a method for producing continental type cheese via the use of optimized processes and bacterial culture design. The disclosure also relates to products prepared by the method as well as bacterial compositions suitable for preparing said products.

### FIELD OF THE DISCLOSURE

The present disclosure relates to the field of cheese products, and in particular making of continental cheese via the use of optimized processes and bacterial culture design.

### BACKGROUND OF THE DISCLOSURE

A general concern of the continental cheese manufacturing process is the acidification profile during production and post-acidification. Indeed, an important parameter in the continental cheese is the final pH of the cheese. After salting, the pH value of the cheese should be above 5.15 and should not decrease further during the ripening stage and storage, to avoid problems with taste or flavors and texture.

Post-acidification is mostly due to the presence of live lactic acid bacteria in the product and residual fermentable sugar. Live lactic acid bacteria continue to convert lactose into lactic acid during storage thereby seriously affecting the taste and texture of the product. Usually, in continental cheese washing steps are included in the manufacturing process to dilute or remove as much lactose as possible before storage. However, the washing step has drawbacks in that it consumes water and energy and takes time. It also generates a diluted whey.

More recently, it has been proposed to reduce or bypass the washing step by using lactose-deficient bacteria or enzymes to decrease the lactose conversion in the curd upon storage.

WO2017/005631 discloses a method and compositions to produce continental cheese in a wash-free process, i.e., where washing is excluded or minimized. The composition comprises mesophilic bacteria with no or very limited acidification power, in particular bacterial strains of the genera Lactococcus, Streptococcus, and Lactobacillus, combined with a specific coagulating enzyme. The process allows to bypass or minimize the washing step while achieving the desired acidification, texture and taste of the cheese produced.

WO2021/239969 discloses a culture for production of continental cheese in a so-called wash-free process. The culture comprises of a mixture of: Lactococcus lactis, lactose deficient Lactococcus lactis and sensitive Streptococcus thermophilus with low post-acidification. The desired flavor and texture profile is obtained while improving yields and resources, in particular for the manufacture of cheese in a Euroblock format (15 kg).

The applicant tried to use the process of WO2021/239969 to produce a continental cheese at scalding temperatures below 38° C. and was surprised to observe that the desired target pH was not reached, and the obtained texture was not useful for continental cheese. The process of WO2021/239969 is therefore not suitable for lower scalding temperatures. Similar suboptimal results were obtained when the process described in WO2021/239969 was carried out to produce smaller cheeses, such as a continental cheese of 8 kg or smaller.

The applicant has therefore judged it necessary to rework the process for manufacturing continental cheese, in a way that would be applicable to any cheese format and/or lower scalding temperatures.

### SUMMARY OF THE DISCLOSURE

To this purpose a universal process seeking to optimize a temperature-controlled ferment composition has been developed for producing continental cheese without requiring a washing step.

In a first aspect the present disclosure provides a method for producing a continental type cheese by providing an initial volume of milk substrate and subjecting said milk substrate to a number of consecutive steps comprising:
(a) Adding a bacterial composition comprising at least 40% of acidifying mesophilic Lactic Acid Bacteria (LAB) and at least 10% of thermophilic LAB, wherein the acidifying mesophilic LAB are characterized by acidifying a milk substrate with 3.5% protein over a period of 6 hours by more than 0.8 pH units when incubated at 35° C. and by more than 0.9 pH units when incubated at 37° C., when inoculated in the milk substrate at 0.01% (w/w);
(b) Optionally pre-ripening the milk substrate between 5-60 min;
(c) Renneting the milk substrate;
(d) Cutting and stirring;
(e) Optionally adding water, wherein the amount of water added is less than 10% of the initial volume of milk substrate;
(f) Scalding;
(g) Optionally pre-pressing the curd;
(h) Molding and pressing; and
(i) Salting;
wherein the size of the cheese is no more than 10 kg and/or the scalding temperature is no more than 38° C.

In the method, control of the acidification profile during and after the fermentation is not based on thermophilic bacteria alone or mesophilic acidifying bacteria alone, but on a balance between thermophiles and acidifying mesophiles. The balance can be adjusted depending on the mass and/or volume of cheese to be produced and the temperature profile to be applied. The method is advantageously devoid of a step of washing the curd.

In a second aspect, the present disclosure relates to a continental type cheese obtained or obtainable by the method.

In a third aspect, the present disclosure relates to bacterial compositions suitable for use in the method.

### DETAILED DESCRIPTION OF THE FIGURES

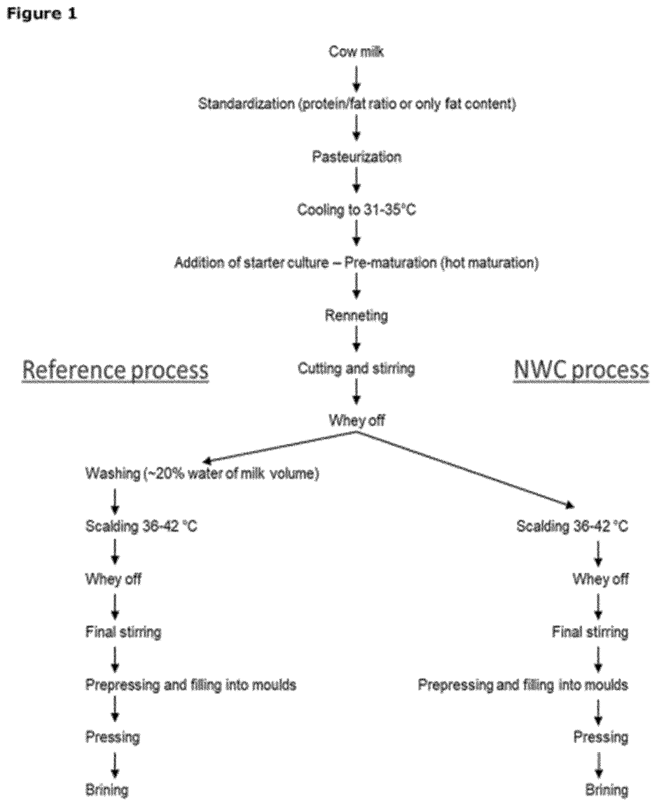

FIG. 1: Schematic example of the cheese manufacturing process for making continental cheese with washing and without washing (WO2021/239969). NWC indicates Non-Washed Curd process.

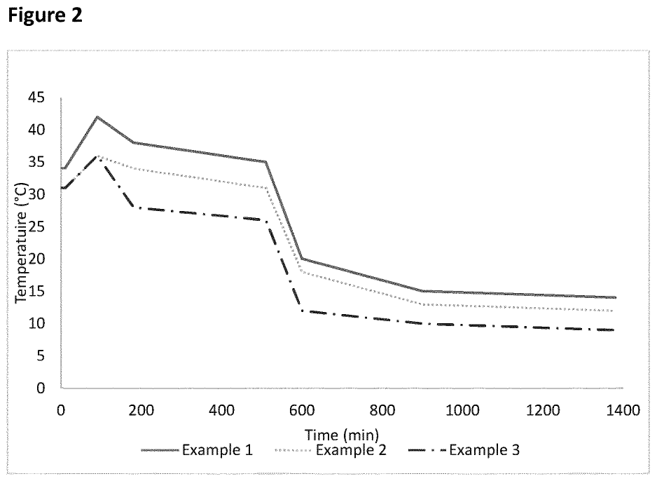

FIG. 2: Temperature profile during cheese making.

### DETAILED DESCRIPTION OF THE DISCLOSURE

### Definitions

The term “milk” is to be understood as the lacteal secretion obtained by milking any mammal, such as cows, sheep, goats, buffaloes, or camels. In a preferred embodiment, the milk is cow's milk. The term “milk substrate” may be any raw and/or processed milk material that can be subjected to fermentation according to the disclosed method. Thus, useful milk substrates include, but are not limited to, solutions/suspensions of any milk or milk like products comprising protein, such as whole or low fat milk, skim milk, buttermilk, reconstituted milk powder, condensed milk, dried milk, whey, whey permeate, lactose, mother liquid from crystallization of lactose, whey protein concentrate, or cream. Obviously, the milk substrate may originate from any mammal, e.g. being substantially pure mammalian milk, or reconstituted milk powder. Preferably, at least part of the protein in the milk substrate is (i) proteins naturally occurring in mammalian milk, such as casein or whey protein or (ii) proteins naturally occurring in plant milk. The term “initial milk substrate volume” means the volume of milk initially procured before commencing the method according to the present disclosure, e.g. the total milk volume in a cheese vat before step (a) of the herein described method.

When used herein, the terms “continental cheese” or “continental type cheese” when used herein refer to semi-hard yellow cheeses, including, but not limited to, Gouda, Edam, Maasdam, Havarti, Danbo, Tilsit or Raclette. The term also includes continental processed cheese.

As used herein, the term “lactic acid bacteria”, abbreviated “LAB”, designates gram-positive, microaerophilic or anaerobic bacteria, which ferment sugar with the production of acids including lactic acid as the predominantly produced acid, acetic acid and propionic acid. The industrially most useful lactic acid bacteria are found within the order “Lactobacillales” which includes Lactococcus spp., Streptococcus spp., Lactobacillus spp., Leuconostoc spp., Pediococcus spp., Brevibacterium spp., Enterococcus spp, and Propionibacterium spp. Lactic acid bacteria, including bacteria of the species Lactobacillus sp. and Streptococcus thermophilus, are normally supplied to the dairy industry either as frozen or freeze-dried cultures for bulk starter propagation or as so-called “Direct Vat Set” (DVS) cultures, intended for direct inoculation into a fermentation vessel or vat for the production of a dairy product, such as a fermented milk product. Such cultures are in general referred to as “starter cultures” or “starters”. The term “starter culture” herein refers to a culture which is a preparation (composition) of one or more bacterial strains (such as lactic acid bacteria strains) to assist the beginning of the fermentation process in preparation of fermented products such as e.g. cheese.

The term “mesophilic” herein refers to microorganisms that thrive best at moderate temperatures (15° C.-35° C.). The industrially most useful mesophilic bacteria include Lactococcus spp. and Leuconostoc spp.

The term “thermophilic” herein refers to microorganisms that thrive best at temperatures above 35° C. The industrially most useful thermophilic bacteria include Streptococcus spp, and Lactobacillus spp.

The terms “lactose-deficient” and “lactose-negative” are used in the context of the present disclosure to characterize LAB which either partially or completely lost the ability to use lactose as a source for cell growth or maintaining cell viability. Such LAB are capable of metabolizing one or more non-lactose carbohydrates selected from sucrose, galactose and/or glucose or another fermentable carbohydrate. Since these carbohydrates are not naturally present in milk in sufficient amounts to support fermentation by lactose-deficient mutants, it is necessary to add these carbohydrates to the milk. Lactose-deficient and partially deficient LAB can be characterized as white colonies on a medium containing lactose as sole source of carbohydrate and X-Gal.

In the context of bacterial compositions, percentages (%) refer to weight percentages.

The term “chymosin” relates to an enzyme of the EC 3.4.23.4 class. The term includes amino acid variants of a naturally-occurring chymosin that have maintained the enzymatic activity. Chymosin has a high specificity and predominantly clots milk by cleavage of a single 105-Ser-Phe-|-Met-Ala-108 bond in kappa-chain of casein. As a side-activity, chymosin also cleaves b-casein primarily between Leu192 and Tyr193. The resulting peptide b(193-209) will be further degraded by proteases to short hydrophobic peptides. An alternative name of chymosin used in the art is rennin.

The term “acidifying mesophilic LAB” refers to bacteria that are characterized by acidifying a milk substrate with 3.5% protein over a period of 6 hours by more than 0.8 pH units when incubated at 35° C. and more than 0.9 pH units when incubated at 37° C., when inoculated in the milk substrate at 0.01% (w/w).

The term “non-acidifying mesophilic LAB” refers to bacteria that are characterized by acidifying a milk substrate with 3.5% protein over a period of 6 hours by less than 0.8 units when incubated at 35° C. and by less than 0.9 pH units when incubated at 37° C., when inoculated in the milk substrate at 0.01% (w/w).

The term “Streptococcus thermophilus with low post-acidification” is defined as a Streptococcus thermophilus strain capable of acidifying a milk with 3.5% protein to a target pH from 0.8 to 1.2 pH units when incubated at 35° C., from 0.9 to 1.4 pH units when incubated at 37° C., and from 1.1 to 1.8 pH units when incubated at 40° C., when inoculated in the milk at 0.01% (w/w) with a start pH of 6.8. By other terms the Streptococcus thermophilus with low post-acidification are in this case defined by the following feature points or functional characteristics:

| Feature point in milk containing 3.5% | | | |
|---|---|---|---|
| protein and an inoculation of 0.01% (w/w) | 35 °C | 37 °C | 40 °C |
| Target pH interval after 6 hours | 5.6-6.0 | 5.4-5.9 | 5.0-5.7 |

### Further Aspects and Embodiments of the Disclosure

As described above, one aspect of the present disclosure is a method for producing a continental type cheese by providing an initial volume of milk substrate and subjecting said milk substrate to a number of consecutive steps comprising:
(a) Adding a bacterial composition comprising at least 40% of acidifying mesophilic Lactic Acid Bacteria (LAB) and at least 10% of thermophilic LAB, wherein the acidifying mesophilic LAB are characterized by acidifying milk substrate with 3.5% protein over a period of 6 hours by more than 0.8 pH units when incubated at 35° C. and by more than 0.9 pH units when incubated at 37° C., when inoculated in the milk substrate at 0.01% (w/w);
(b) Optionally pre-ripening the milk substrate between 5-60 min;
(c) Renneting the milk substrate;
(d) Cutting and stirring;
(e) Optionally adding water, wherein the amount of water added is less than 10% of the initial milk substrate volume, such as e.g., from 0.1% to 10%, 0.1% to 8%, 0.1% to 6%, 0.1% to 4%, or 0.1% to 2% of the initial milk substrate volume;
(f) Scalding;
(g) Optionally pre-pressing the curd;
(h) Molding and pressing; and
(i) Salting;
wherein the size of the cheese is no more than 10 kg and/or the scalding temperature is no more than 38° C.

A non-limiting flow chart for producing continental cheese with and without washing can be seen in FIG. 1.

In one embodiment, the milk substrate that is provided comprises from 3.1% to 4.0% of protein, such as from 3.2% to 3.8% of protein or from 3.4% to 3.6% of protein.

In one embodiment, the acidifying mesophilic LAB are characterized by acidifying a milk substrate with 3.5% protein over a period of 6 hours by between 0.8 and 1.2 pH units when incubated at 35° C. and by between 0.9 and 1.4 pH units when incubated at 37° C., when inoculated in the milk substrate at 0.01% (w/w).

In one embodiment, the acidifying mesophilic LAB are characterized by acidifying a milk substrate with 3.5% protein over a period of 6 hours by between 0.8 and 1.0 pH units when incubated at 35° C. and by between 0.9 and 1.2 pH units when incubated at 37° C., when inoculated in the milk substrate at 0.01% (w/w).

In one embodiment, the bacterial composition added to the milk in step (a) comprises from 40% to 90%, from 45% to 80%, from 50% to 70% or from 50% to 60% of acidifying mesophilic LAB.

In one embodiment, the bacterial composition comprises lactose-deficient LAB, such as mesophilic lactose-deficient LAB and/or thermophilic lactose-deficient LAB. In one embodiment, the bacterial composition comprises lactose-positive (i.e., lactose-metabolizing) LAB, such as mesophilic lactose-positive LAB and/or thermophilic lactose-positive LAB. The bacterial composition may also comprise both lactose-deficient LAB and lactose-positive LAB.

In one embodiment, the acidifying mesophilic LAB comprise Lactococcus lactis subsp. lactis and/or Lactococcus lactis subsp. cremoris.

In one embodiment, the bacterial composition comprises from 10% to 50%, from 15% to 40% or from 10% to 30% of thermophilic LAB.

In one embodiment, the thermophilic LAB comprise a strain of the species Streptococcus thermophilus.

In one embodiment, the thermophilic LAB comprise a strain of Streptococcus thermophilus with low post-acidification.

In one embodiment, the thermophilic LAB comprise a strain of Streptococcus thermophilus with low post-acidification, wherein the strain is lactose-deficient.

In one embodiment, the bacterial composition comprises or further comprises non-acidifying mesophilic LAB, preferably from 5% to 30%, from 10% to 30%, from 10% to 25% or from 15% to 20% of non-acidifying mesophilic LAB.

In one embodiment, the non-acidifying mesophilic LAB comprise Lactococcus lactis and/or Leuconostoc. Lactococcus lactis may be selected from the group of subspecies containing: lactis, cremoris, and biovar diacetylactis.

In one embodiment, the non-acidifying mesophilic LAB comprise Lactococcus lactis spp. lactis and/or Lactococcus lactis spp. cremoris. In one embodiment, the non-acidifying mesophilic LAB are lactose-deficient LAB.

In one embodiment, the bacterial composition comprises a ratio of non-acidifying mesophilic LAB to acidifying mesophilic LAB from 1:1 to 1:5, from 1:2 to 1:4, from 1:2.5 to 1:3.5, or from 1:3.2 to 1:3.3.

In one embodiment, the bacterial composition comprises from 50% to 60% of acidifying mesophilic LAB, from 20% to 30% of thermophilic LAB, and from 10% to 30% non-acidifying mesophilic LAB. Such a bacterial composition is particularly suitable for methods wherein the temperature during the scalding step is from 36° C. to 38° C.

In one embodiment, the bacterial composition comprises from 40% to 90% of acidifying mesophilic LAB, from 10% to 30% of thermophilic LAB, and from 10% to 30% non-acidifying mesophilic LAB.

In one embodiment, the bacterial composition comprises from 50% to 70% of acidifying mesophilic LAB, from 10% to 30% of thermophilic LAB, and from 10% to 30% non-acidifying mesophilic LAB. Such a bacterial composition is particularly suitable for methods wherein the temperature during the scalding step is from 34° C. to 36° C.

In one embodiment, the bacterial composition comprises from 50% to 60% of acidifying mesophilic LAB, wherein the acidifying mesophilic LAB are Lactococcus lactis, from 20% to 30% of thermophilic LAB, wherein the thermophilic LAB are S. thermophilus with low post-acidification and from 10% to 20% of non-acidifying LAB, wherein the non-acidifying LAB are lactose-deficient Lactococcus lactis. In one embodiment, the acidifying mesophilic LAB are bacteria that have a maximum acidification rate of more than or equal to 0.40 pH units per hour in B-milk (B-milk is Reconstituted Skimmed Milk (9.5%) heated to 99° C. for 30 min) and has a maximum acidification rate of more than 0.05 pH units per hour in B-milk added 4% salt (NaCl).

In one embodiment, the bacterial composition comprises one or more of the following bacterial strains: DSM 24649 [WO2012/175752]; DSM 26564 [WO2015/067808]; and/or DSM 26565 [WO2015/067808].

The composition of the present disclosure may be provided in several forms. It may be a powder, pellets or tablets. It may be a frozen form, dried form, freeze dried form, or liquid form. Thus, in one embodiment the composition is in frozen, dried, freeze-dried or liquid form. In one embodiment the bacterial composition may be supplied as frozen pellets, such as e.g., as a Direct Vat Set culture.

In a further aspect, the composition of the present disclosure contains or comprises an ammonium salt (e.g. an ammonium salt of organic acid (such as ammonium formate and ammonium citrate) or an ammonium salt of an inorganic acid) as a booster (e.g. growth booster or acidification booster) for bacterial cells, such as cells belonging to the species S. thermophilus, e.g. (substantial) urease negative bacterial cells. The term “ammonium salt”, “ammonium formate”, etc., should be understood as a source of the salt or a combination of the ions. The term “source” of e.g. “ammonium formate” or “ammonium salt” refers to a compound or mix of compounds that when added to a culture of cells, provides ammonium formate or an ammonium salt. In some embodiments, the source of ammonium releases ammonium into a growth medium, while in other embodiments, the ammonium source is metabolized to produce ammonium. In some preferred embodiments, the ammonium source is exogenous. In some particularly preferred embodiments, ammonium is not provided by the dairy substrate. It should of course be understood that ammonia may be added instead of ammonium salt. Thus, the term ammonium salt comprises ammonia (NH3), NH4OH, NH4+, and the like. In some embodiments, the bacterial composition comprises or further comprises from 1% to 20% (w/w) of a booster.

Step (a) may be preceded by heating the milk to around 72° C.-74° C. for 5-60 sec and/or cooling the milk to 31-35° C. The rennet applied in step (c) may preferably be a chymosin with low unspecific proteolysis such as e.g., camel chymosin or bovine chymosin.

The method of present disclosure may further comprise a drying step between step (d) and (f) wherein the curd is dried before molding to reduce wet matter and control the moisture content of the cheese.

In some embodiments of the disclosed method, step (e) is not performed, and thus no water is added to the milk composition (curd) prior to scalding.

In some embodiments of the disclosed method, water is added in step (e), wherein the amount of water added is less than 10% of the initial milk substrate volume, such as e.g., from 0.1% to 10%, 0.1% to 8%, 0.1% to 6%, 0.1% to 4%, or 0.1% to 2% of the initial milk substrate volume.

In some embodiments of the disclosed method, step (e) comprises a washing step, wherein water corresponding to less than 10% of the initial milk substrate volume is added after the washing, liquid is removed. In some embodiments of the disclosed method, step (e) is performed, but the amount of water added is less than 10% of the initial milk substrate volume, such as less than 9%, less than 8%, less than 7%, less than 6%, less than 5%, less than 4%, less than 3%, less than 2%, or less than 1% of the initial milk substrate volume.

In some embodiments, the scalding step (f) is performed at a temperature below 38° C., such as below 37° C., below 36° C., below 35° C. or below 34° C. In one embodiment, the scalding temperature is no more than 37° C., no more than 36° C., no more than 35° C. or no more than 34° C. In some embodiments, the scalding step is performed at a temperature from 36° C. to 38° C. or from 34° C. to 36° C.

In some embodiments, the salting step (i) may be dry salting or brining.

The disclosed method may be used for the manufacture of several cheese types including, but not limited to, the continental cheese types selected from: Gouda, Edam, Maasdam, Havarti, Danbo, Tilsit or Raclette. The cheese may be in Euroblock format.

The disclosed method may be used for the production of a continental type cheese having a weight below 10 kg, below 9 kg, below 8 kg, below 7 kg, below 6 kg, below 5 kg, below 4 kg, below 3 kg, below 2 kg, or below 1 kg. In one embodiment, the method is for the production of a continental type cheese having a weight from 0.001 to 10 Kg, from 0.01 to 9 Kg, from 0.1 to 8 Kg, from 1 to 7 Kg, from 2 to 6 Kg, from 3 to 5 Kg, from 4 to 10 Kg, from 5 to 9 Kg, or from 6 to 8 Kg.

In a further aspect, the disclosure relates to a continental type cheese obtained or obtainable by the disclosed method as described herein. In one embodiment, the cheese is Gouda, Edam, Maasdam, Havarti, Danbo, Tilsit or Raclette. The cheese may be in Euroblock format.

In one embodiment, the cheese has a weight below 10 kg, below 9 kg, below 8 kg, below 7 kg, below 6 kg, below 5 kg, below 4 kg, below 3 kg, below 2 kg, or below 1 kg. In one embodiment, the cheese has a weight from 0.001 to 10 Kg, from 0.01 to 9 Kg, from 0.1 to 8 Kg, from 1 to 7 Kg, from 2 to 6 Kg, from 3 to 5 Kg, from 4 to 10 Kg, from 5 to 9 Kg, or from 6 to 8 Kg.

In a further aspect, the disclosure relates to a bacterial composition suitable for use in the disclosed method as described herein, wherein the bacterial composition comprises at least 40% of acidifying mesophilic lactic acid bacteria and at least 10% of thermophilic lactic acid bacteria. The bacterial composition may be composed as disclosed supra and further have any one of the features as described above. The composition may be a mixture or as a kit-of-parts.

In one embodiment, the bacterial composition comprises one or more of the following bacterial strains: DSM 24649; DSM 26564; and/or DSM 26565.

All references, articles, publications, patents, patent publications, and patent applications cited herein are incorporated by reference in their entireties for all purposes.

### Examples

### Example 1—Continental Cheese, Eurobloc Format (15 kg) with High Scalding Temperature, without Curd Washing and the Culture Design from Patent WO2021/239969

A continental Eurobloc (15 kg) cheese (Gouda) was produced according to a process without curd washing step essentially as described in WO2021/239969, with a scalding temperature of 42° C. (Table 1).

The starter culture used was a mixture of Lactococcus lactis, lactose-deficient Lactococcus lactis and Streptococcus thermophilus with low post-acidification (Table 2), Chr. Hansen® (Denmark) as described in WO2021/239969. The temperature profile of the method is described in Table 1 and FIG. 1.

From renneting to scalding, the temperature was increased from 34 C° to 42° C. After molding, the temperature decreased slowly from 42° C. to arrive at 38° C. at the pressing step and 35° C. in the acidification chamber. This kinetic allows the production of lactic acid from culture until salting. At brining, the pH of the cheese was 5.80. Due to the residual of lactose (effect of the no wash curd process), the pH dropped between brine and day 1 to 5.38 and showed a drop until 5.25 during the first month of ripening (Table 3). These results were satisfactory for the production of this type of cheese.

### Example 2—Continental Cheese, Eurobloc Format (15 kg) with Lower Scalding Temperature, without Curd Washing and the Culture Design from Patent WO2021/239969

A continental Eurobloc (15 kg) cheese (Gouda) was produced according to a process without curd washing step essentially as described in WO2021/239969, with a scalding temperature of 36° C. (Table 1).

The starter culture used was the same as used in Example 1, i.e. a mixture of Lactococcus lactis, lactose-deficient Lactococcus lactis and Streptococcus thermophilus with low post-acidification (Table 2), Chr. Hansen® (Denmark) as described in WO2021/239969. The temperature profile of the method is described in Table 1 and FIG. 1.

From renneting to scalding, the temperature was increased from 31 C° to 36° C. After molding, the temperature decreased slowly from 36° C. to arrive at 34° C. at the pressing step and 31° C. in the acidification chamber. Surprisingly, this kinetic, i.e. earlier reduction to low temperatures, did not allow enough growth of the culture and therefore not enough production of lactic acid from culture before brine. At brining the pH of the cheese was 5.99. Even though the cheese contained some residual lactose (effect of the no wash curd process), the pH was 6.02 at day 1 and thus did not drop to the target pH (5.15-5.40) between brine and day 1. Furthermore, the pH did not drop during the first month of ripening (Table 3). Thus, surprisingly, the starter culture was not suitable for a process with a scalding temperature of 36° C.

### Example 3—Continental Cheese, Smaller Format (<1 kg) with Lower Scalding Temperature, without Curd Washing and the Use of the New Culture Design

A continental cheese (Gouda) of less than 1 kg was produced according to a process without curd washing step, with a scalding temperature of 36° C. (Table 1).

In this example, the starter culture was a mixture of Lactococcus lactis (including strains DSM 24649; DSM 26564; and DSM 26565), lactose-deficient Lactococcus lactis and Streptococcus thermophilus with low post-acidification, with an increased proportion of acidifying mesophilic LAB (Table 2), Chr. Hansen® (Denmark). The temperature profile of the method is described in Table 1 and FIG. 1.

From renneting to scalding, the temperature increases from 31° C. to 36° C. After molding, the temperature decreases quickly from 36° C. to arrive at 28° C. at the pressing step and 26° C. in the acidification chamber. Despite this earlier reduction to low temperatures, the specific culture design used in this Example 3 allows a very good growth of the culture. At brine, the pH of the cheese was 5.32 and even though the cheese contained residual of lactose (effect of the no wash curd process), the pH stayed relatively stable over the time, with a small drop between brine and day 1 but this pH increased after, due to ripening process (Table 3). Thus, using the modified starter culture satisfactory results were obtained.

The method of Example 3 was also carried out for an 8 kg format Gouda cheese and a similar satisfactory pH profile was obtained (data not shown).

TABLE 1
Temperature profile of the methods.

| | | Example 1 | Example 2 | Example 3 |
|---|---|---|---|---|
| | | High scalding | Low scalding | Low scalding |
| | | Temp | Temp | Temp |
| | | (°C) | (°C) | (°C) |
| | | Cheese format | | |
| | | Eurobloc | Eurobloc | Lower than |
| | | 15 kg | 15 kg | 1 kg |
| PROCESS STEP and | Time | Scalding temperature (°C) | | |
| TEMPERATURE (°C) | (min) | 42 | 36 | 36 |
| Inoculation | 0 | 34 | 31 | 31 |
| Renneting | 10 | 34 | 31 | 31 |
| Molding | 90 | 42 | 36 | 36 |
| Pressing | 180 | 38 | 34 | 28 |
| Acidification chamber | 510 | 35 | 31 | 26 |
| Brine | 600 | 20 | 18 | 12 |
| Storage | 900 | 15 | 13 | 10 |
| Coating | 1380 | 14 | 12 | 9 |

TABLE 2
Composition of the bacterial cultures.

| | Example | Example | Example |
|---|---|---|---|
| | 1 | 2 | 3 |
| Thermophilic LAB | 27% | 27% | 28% |
| (S. thermophilus with low post-acidification) | | | |
| Non-acidifying mesophilic LAB | 67% | 67% | 17% |
| (lactose-deficient Lactococcus lactis) | | | |
| Acidifying mesophilic LAB | 6% | 6% | 55% |
| (Lactococcus lactis) | | | |

TABLE 3
Post-acidification (pH) profile using different combinations of culture.

| | Example 1 | Example 2 | Example 3 |
|---|---|---|---|
| Renneting | 6.63 | 6.55 | 6.52 |
| Brining | 5.80 | 5.99 | 5.32 |
| Storage 24 hours | 5.38 | 6.02 | 5.22 |
| Storage 2 weeks | 5.25 | 6.01 | 5.39 |
| Storage 4 weeks | 5.27 | 5.99 | 5.41 |
| Storage 6 weeks | 5.32 | 6.09 | 5.37 |

1. A method for producing a continental type cheese by providing an initial volume of milk substrate and subjecting said milk substrate to a number of consecutive steps comprising:
(a) Adding a bacterial composition comprising at least 40% of acidifying mesophilic Lactic Acid Bacteria (LAB) and at least 10% of thermophilic LAB, wherein the acidifying mesophilic LAB are characterized by acidifying a milk substrate with 3.5% protein over a period of 6 hours by more than 0.8 pH units when incubated at 35° C. and by more than 0.9 pH units when incubated at 37° C., when inoculated in the milk substrate at 0.01% (w/w);
(b) Optionally pre-ripening the milk substrate between 5-60 min;
(c) Renneting the milk substrate;
(d) Cutting and stirring;
(e) Optionally adding water, wherein the amount of water added is less than 10% of the initial milk substrate volume;
(f) Scalding;
(g) Optionally pre-pressing the curd;
(h) Molding and pressing; and
(i) Salting;
wherein the size of the cheese is no more than 10 kg and/or the scalding temperature is no more than 38° C.

2. The method according to claim 1, wherein the bacterial composition comprises between 40% and 90%, between 45% and 80% or between 50% and 70% of acidifying mesophilic LAB.

3. The method according to claim 2, wherein the acidifying mesophilic LAB comprise Lactococcus lactis subsp. lactis and/or Lactococcus lactis subsp. cremoris.

4. The method according to claim 1, wherein the bacterial composition comprises between 10% and 50%, between 15% and 40% or between 10% and 30% of thermophilic LAB.

5. The method according to claim 4, wherein the thermophilic LAB comprise a strain of the species Streptococcus thermophilus.

6. The method according to claim 1, wherein the bacterial composition further comprises non-acidifying mesophilic LAB, wherein the bacterial composition comprises from 5% to 30%, from 10% to 25% or from 15% to 20% of non-acidifying mesophilic LAB.

7. The method according to claim 6, wherein the non-acidifying mesophilic LAB comprise Lactococcus lactis and/or Leuconostoc.

8. The method according to claim 7, wherein the Lactococcus lactis is selected from the group of subspecies containing: lactis, cremoris, and biovar diacetylactis.

9. The method according to claim 1, wherein the bacterial composition comprises from 40% to 90% of acidifying mesophilic LAB, from 10% to 30% of thermophilic LAB, and from 10% to 30% non-acidifying mesophilic LAB.

10. The method according to claim 1, wherein one or more acidifying mesophilic LAB are bacteria that have a maximum acidification rate of more than or equal to 0.40 pH units per hour in B-milk and has a maximum acidification rate of more than 0.05 pH units per hour in B-milk added 4% salt (NaCl).

11. The method according to claim 1, wherein the bacterial composition comprises strain DSM 24649, strain DSM 26564, and/strain or DSM 26565.

12. The method according to claim 1, wherein the scalding temperature is no more than 37° C., no more than 36° C., no more than 35° C. or no more than 34° C.

13. The method according to claim 1, wherein the method is for the production of a continental type cheese having a weight from 0.001 to 10 Kg, from 0.01 to 9 Kg, from 0.1 to 8 Kg, from 1 to 7 Kg, from 2 to 6 Kg, from 3 to 5 Kg, from 4 to 10 Kg, from 5 to 9 Kg, or from 6 to 8 Kg.

14. A continental type cheese obtained or obtainable by the method of claim 1.

15. The cheese according to the claim 14, wherein the cheese is Gouda, Edam, Maasdam, Havarti, Danbo, Tilsit or Raclette.

16. A bacterial composition suitable for use in the method of claim 1, wherein the bacterial composition comprises at least 40% of acidifying mesophilic LAB and at least 10% of thermophilic LAB.

17. (canceled)

18. The bacterial composition according to claim 16, comprising strain DSM 24649, strain DSM 26564 and/or strain DSM 26565.
